(19) United States
(12) Patent Application Publication
(10) Pub. No.: US 2014/0235974 A1
(43) Pub. Date: Aug. 21, 2014

(54) INTEGRATED CALIBRANT MEASUREMENT SYSTEM FOR ANALYTE SENSORS

(71) Applicant: EDWARDS LIFESCIENCE CORPORATION, Irvine, CA (US)

(72) Inventors: Michael Higgins, Huntington Beach, CA (US); Jennifer L. Wilbur, Newport Beach, CA (US); Paul S. Van Wieren, Costa Mesa, CA (US)

(73) Assignee: Edward Lifesciences Corporation, Irvine, CA (US)

(21) Appl. No.: 14/351,515

(22) PCT Filed: Oct. 10, 2012
(86) PCT No.: PCT/US2012/059541

§ 371 Date: Apr. 11, 2014

Related U.S. Application Data
(60) Provisional application No. 61/545,861, filed on Oct. 11, 2011.

(51) Int. Cl. A61B 5/1495; A61B 5/00; A61B 5/1468; A61B 5/15; A61B 5/1455

(52) U.S. Cl. CPC A61B 5/1495; A61B 5/150992; A61B 5/1455; A61B 5/1468; A61B 5/7278
USPC 600/322; 600/309; 600/345

(57) ABSTRACT

Embodiments of the present invention provide a blood analyte sensing system (10) that includes an integral calibrant concentration measurement system. The blood analyte sensing system includes a blood sensor (22), a calibration system and the calibrant concentration measurement system. The calibrant concentration measurement system is connected in communication with both the calibration system and the blood sensor. The calibration concentration measurement system includes a calibrant sensor (20) configured to measure a calibrant concentration in response to exposure to the calibrant. The calibration concentration measurement system is also configured to determine the analyte concentration of the patient's blood using the calibrant concentration, calibration signal and the blood signal.

### FIELD OF THE INVENTION

This invention relates to analyte sensing systems, and more particularly to blood analyte sensing systems that use calibrants to improve accuracy and sensitivity.

### BACKGROUND OF THE INVENTION

Blood glucose monitoring systems use a calibrant solution to continually calibrate an electrochemical sensor. For optimal system performance, this calibrant solution is often supplied to the sensor at physiological concentrations, such as 190-210 mg/dL. However, generally the only dextrose solutions available in a clinical environment are at relatively high concentrations (e.g., D5 at over 4,000 mg/dL).

In order to provide the current systems with a dextrose solution at the proper concentration, then, a healthcare professional prepares a diluted glucose solution on a per-unit basis. Currently, this is typically performed in the pharmacy by injecting a bolus of about 2.4 mL of D50 into a 500 mL heparinized saline bag to arrive at a solution of roughly 200 mg/dL. After preparing the bag, the pharmacy is also responsible for quantifying its dextrose concentration.

Despite the apparent simplicity of this approach, many pharmacies do not have readily-available equipment that is able to quantify the calibrant concentration. Many glucose analyzers are for whole blood glucose measurements. Pharmacies may also lack scales with sufficient precision to measure the calibrant components. Further, the labor costs and time delays generally make a pharmacy-based creation of a calibrant inconvenient and expensive.

Improvements in the calibration of blood analyte sensors, therefore, are needed to promote easy and convenient measurement of blood analytes that are of interest to medical personnel.

### SUMMARY OF THE INVENTION

Embodiments of the present invention overcome the problems of the prior art by providing a blood analyte sensing system that includes an integral calibrant concentration measurement system. The blood analyte sensing system includes a blood sensor, a calibration system and the calibrant concentration measurement system. The blood sensor is configured to generate a blood signal in response to exposure to the patient's blood. The calibration system is connected in communication with the blood sensor. And, it is configured to record a calibration signal from the blood sensor in response to exposure to the calibrant. The calibrant concentration measurement system is connected in communication with both the calibration system and the blood sensor. The calibration concentration measurement system includes a calibrant sensor configured to measure a calibrant concentration in response to exposure to the calibrant. The calibration concentration measurement system is also configured to determine the analyte concentration of the patient's blood using the calibrant concentration, calibration signal and the blood signal.

In another embodiment, the calibrant concentration measurement system is connected in fluid communication with the blood sensor and the calibrant passes through the calibrant concentration measurement system on its way to the blood sensor. In particular, the measurement of the calibrant concentration occurs prior to recording of the calibration signal. This enables recording of the calibration signal and measurement of the calibrant concentration can occur in real-time or substantially simultaneously.

In another embodiment, the calibrant concentration measurement system is connected in fluid communication with the blood sensor and is configured to receive and flow calibrant over the calibrant sensor and the blood sensor. The blood analyte sensing system may also include a calibrant source, or sources, that are a range of unknown or inexactly known concentrations. With two or more sources, once source may have a higher concentration (such as D5) of calibrant than the other source (which may have zero concentration, such as buffered saline), and the two are mixed together in real time, or at time of use, before being routed through the calibrant concentration measurement system.

In another embodiment, the blood sensor is consumable and the calibration system is configured to expose the blood sensor to the calibrant at regular intervals over the life of the blood sensor. For example, the blood sensor may have a life of less than two weeks, or 72 hours, and the regular intervals are 10 minutes or less.

In another embodiment, the blood sensing system includes a fluid control system connected in communication with both the calibration system and the calibrant concentration measurement system. The fluid control system is configured to control exposure of the blood sensor and the calibrant sensor to the calibrant, and of the blood sensor to the patient's blood. For example, the fluid control system may include a pump connected to a fluid column that includes an upper calibrant portion and a lower blood portion separated by a transition region. In particular, the fluid column extends through the pump, and the pump is configured to move the transition region above and below the sensor in cycles. The calibrant sensor in this embodiment may be positioned upstream of the blood sensor, along the fluid column, and the pump further configured to stop movement of the transition region before it reaches the calibrant sensor.

In other embodiments, the calibrant sensor may use one or more of polarization, refractometry, spectroscopy, density, viscosity, electrical impedance or specific heat of the calibrant to determine the calibrant concentration.

One embodiment includes a polarimeter as the calibrant sensor, the polarimeter including a light source, a first polarizer and a light detector. The light source, for example, may include a LED. The LED may be a green or a blue light-emitting diode emitting light in the range of 405 nm to 525 nm wavelengths. Other or multiple wavelengths can be employed for better differentiation of interferants and noise. For example, red at approximately 633 nm could be additionally employed for better results.

The first polarizer may include a rotator that's configured to rotate the polarized light for AC modulation. The first polarizer is positioned on side of the calibrant in the path of emitted light, while the light detector is positioned on the other side of the calibrant. A second polarizer may be positioned on the side of the light detector, wherein the second polarizer includes a rotator and is configured to rotate to compensate for calibrant-induced rotation. This rotation may be controlled by a controller that communicates with the light detector to minimize detected light exiting the second polarizer.

Embodiments of the present invention have a calibrant concentration measurement with a relatively low error for a physiologically useful 0-100 mg/dL range of calibrant concentration using a path length of less than 5 cm. Other accurate ranges are centered around 100 mg/dL, such as 90-110 or 80-120 mg/dL.

In another embodiment, the polarimeter may include an amplifier that is configured to provide a sinusoidal drive signal to the Faraday modulator. And, the amplifier may include a digital lock-in amplifier configured to lock in the detector output to the frequency of the drive signal provided to the Faraday modulator.

In another embodiment, the polarimeter may include a sample chamber through which there is continuous calibrant flow. And, the polarimeter may be configured to recalibrate itself based on measurements of a sample of known concentration.

Other embodiments of the present invention include methods of, and computer programs for, implementing the systems described above.

These and other features and advantages of the present invention will become more readily apparent to those skilled in the art upon consideration of the following detailed description and accompanying drawings, which describe both the preferred and alternative embodiments of the present invention.

### BRIEF DESCRIPTION OF THE DRAWINGS

FIG. 1 is a perspective view of a blood analyte measurement system of one embodiment of the present invention;

FIG. 2 is a schematic of a blood analyte measurement system of another embodiment of the present invention wherein a pump module is downstream of a mixing module and a measurement module is downstream of the pump module;

FIG. 3 is a schematic of a blood analyte measurement system of another embodiment of the present invention wherein a measurement module is downstream of a mixing module and a pump module is downstream of the measurement module;

FIG. 4 is a schematic of a blood analyte measurement system of another embodiment of the present invention wherein a mixing module is in a flow control housing, a measurement module is downstream of the mixing module, and a pump module is downstream of the measurement module;

FIG. 5 is a schematic of a blood analyte measurement system of another embodiment of the present invention wherein a mixing module is in a flow control housing, a pump module is downstream of the mixing module, and a measurement module is downstream of the pump module;

FIG. 6 is a schematic of a blood analyte measurement system of another embodiment of the present invention having two rotary pinch valves;

FIG. 7 is a schematic of a polarimetry system of another embodiment of the present invention using a liquid crystal polarizing rotator;

FIG. 8 is a schematic of a polarimetry system of another embodiment of the present invention using a Faraday modulator and compensator;

FIG. 9 is a graph of a sinusoidal drive signal (top) and a corresponding photodetector output signal (bottom) for a perfectly nulled polarimetry system of FIG. 8;

FIG. 10 is a graph of an output signal of the polarimetry system of FIG. 8 before nulling occurs;

FIGS. 11 and 12 are graphs of error testing results of the polarimetry system of FIG. 8; and

FIG. 13 is a schematic of generation and amplification of a reference signal.

### DETAILED DESCRIPTION OF THE INVENTION

The present invention now will be described more fully hereinafter with reference to specific embodiments of the invention. Indeed, the invention can be embodied in many different forms and should not be construed as limited to the embodiments set forth herein; rather, these embodiments are provided so that this disclosure will satisfy applicable legal requirements. As used in the specification, and in the appended claims, the singular forms “a”, “an”, and “the”, include plural referents unless the context clearly dictates otherwise. The term “comprising” and variations thereof as used herein are used synonymously with the term “including” and variations thereof and are open, non-limiting terms.

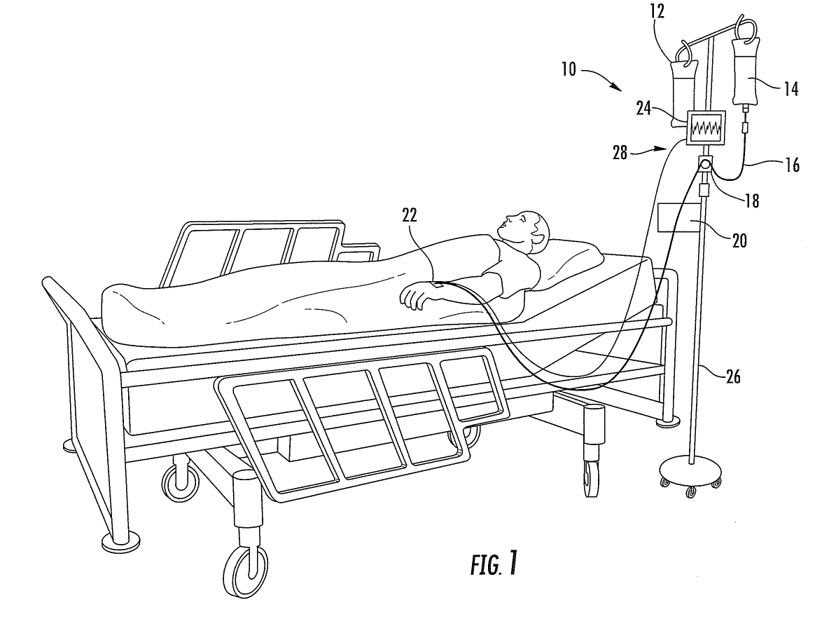

Embodiments of the present invention include an analyte measurement system 10 having a calibrant source 12, a diluent source 14, fluid supply lines 16, a pump 18, a calibrant sensor 20, a blood analyte sensor 22 and a monitor 24, as shown in FIG. 1. Generally, the analyte measurement system 10 allows relatively (for the purposes of calibration in an analyte measurement system) imprecise or inaccurate combination of calibrant from the calibrant source 12 and diluent from the diluent source 14 with accurate, separate sensing of the diluted calibrant solution by the calibrant sensor 20 before it is used to calibrate the blood analyte sensor 22. Advantageously, this real-time (or near real-time) sensing of the calibrant solution avoids the need for a custom formulated supply of calibrant. It also avoids the need to tightly control flow and combination (e.g., mixing) of the separate calibrant and diluent sources, 12 and 14. Also, it ensures that an accurate concentration of the analyte in the calibrant is known right before its use on the blood analyte sensor 22.

The term “real time” as used herein is on the order of 10 minutes, depending upon cycle times, and may be even within seconds, depending upon the rate of calibrant delivery. Generally, real time does not include the delay associated with the times needed for preparation of a bag at a hospital pharmacy.

The calibrant source 12 in one embodiment includes a bag of standard calibrant solution, such as D5 or D50 for glucose sensors. Calculations for concentration may also be adjusted for the use of hydrous dextrose as a component of the D5 or D50W which are normally primarily dextrose monohydrate. The calibrant source in one embodiment is suspended on a pole 26, which enables a gravity drip feed downward into an attached one of the fluid lines 16, as shown in FIG. 1. Although illustrated as a standardized bag of liquid, the calibrant source could be from a liquid, gel, solid (e.g., sugar pills) or other composition and supplied through lines, by pumps or part of a stream of the diluent source passed over the solids, etc. Generally, however, embodiments of the present invention are advantageous in circumstances where their use obviates the need for tightly controlled composition and supply of the calibrant from the calibrant source 12. It is the inventors' observation that controlling concentration with accuracy is more difficult in most circumstances than measuring the resulting concentration with accuracy.

The diluent source 14 in one embodiment includes a bag of standard buffered saline and may also include heparin or some anticoagulant. Like the calibrant source 12, the diluent source 14 is suspended from the pole 26, which enables a gravity drip feed downward into an attached one of the fluid lines 16, as shown in FIG. 1. Although illustrated as a standardized bag of liquid, the diluent source could be from some other type of container, a fixed line, etc., as long as the diluent is configured to dilute the calibrant source 12 to a lower concentration.

In other embodiments, a plurality of sources may be employed with different combinations of diluent and calibrant depending upon the anticipated mixture of these components downstream at the calibrant sensor 20. Multiple sources may also be used if long time periods are expected to elapse before changing the source or if large amounts of calibrant are being employed. Also, the calibrant or diluent sources 12, 14 may be the source for a flushing cycle, which would drive up their depletion or change their composition. Further, in another embodiment, the calibrant may already be mixed in its final form available from a source, such as a bag, but not have had an accurate or precise prior determination of its composition. Also, the calibrant concentration if known with accuracy already may be checked or confirmed merely as an additional safety measure.

Regardless, returning to FIG. 1, the fluid supply lines 16 extend down from the calibrant source 12 and the diluent source 14 into the pump 18. Although not shown in detail, at some point in the illustrated embodiment the two fluid supply lines leading from the sources 12, 14 combine to mix together the calibrant and diluent into the final calibrant solution composition. For example, one of the fluid lines may be nested into another or both may contribute to a drip chamber in which the two source fluids mix before, during or after being urged toward the blood analyte sensor 22. This mixing may also be facilitated by the action of the pump 20.

In the illustrated embodiment, the pump 20 is a rotary pinch valve or peristaltic pump that employs one or more rotating cams to apply a rolling pinch or compression to the outside of one or both of the fluid supply lines 16. When combined with the head generated by the elevation of the calibrant and diluent sources 12, 14, the pump 18 controls flow and positioning of a fluid column that extends down through the fluid supply lines 16 into the bloodstream of the patient. Operation of the pump 20 is enabled by hardware, software, or firmware housed in the monitor 24, which controls a motor that turns or drives the cams. Generally, therefore, this system operates collectively as a fluid or flow control system 28.

As they near the patient, the fluid supply lines 16 of some embodiments are consolidated into a single monitor line that ends at or within, or is supported by, a catheter. For example, the catheter may reside in the patient and provide access to the patient's blood flow, from which can be drawn samples by the pump 20. The blood samples are drawn up over the blood analyte sensor 22 for determination of blood analyte levels at that instant. Alternatively, the blood analyte sensor 22 may extend out of the catheter and into the patient's blood stream, in which case the fluid supply lines 16 may only function to flush or calibrate the blood analyte sensor 22. In another embodiment, the blood analyte sensor 22 may be in its own dedicated catheter that is sleeved into an existing catheter, such as a central venous catheter (CVC) or a peripherally inserted central catheter (PICC). Regardless, these components can also be considered to be part of the flow control system 28.

As an example of flow control by the fluid control system 28, the blood analyte sensor 22 may be subjected to varying cycles of exposure to the calibrant, diluents, and patient blood. For example, a flush cycle may include directing substantial amounts of diluent from the diluent source 14 containing heparin or other anticoagulants over the blood analyte sensor 22 to free it of incipient thrombosis. A calibration cycle may include a controlled advancement of the calibrant portion of the fluid column first over the calibrant sensor 20 for analyte concentration determination and then over the blood analyte sensor 22 for calibration of the sensor 22. Also, an analyte sensing cycle may include rolling the rotary pinch pump 18 in the opposite direction against the head of the hanging bags 12, 14 to draw blood up from the patient over the blood analyte sensor 22 for determination of analyte concentration. The region between the undiluted blood and calibrant is a transition region that is controlled to pass the blood analyte sensor during reciprocating flush, calibration and draw sequences. A combined calibration, draw and flush cycle, in one embodiment for example, could last on the order of 5 to 10 minutes and extend over a 72 hour period. Embodiments of the present invention preferably can sustain continuous calibrant concentration measurement in cycles over such a period.

It should be noted that flow of the calibrant or blood samples over the calibrant sensor 20 and the blood analyte sensor 22 can be controlled by other combinations of components that form a fluid control system 28 and still fall within the scope of the present invention. For example, the fluid control system 28 may include different types of pumps that generate head and obviate the need for hanging bags. Also, other fluid communicators may be used to connect the calibrant and diluent sources 12, 14 to the calibrant sensor 20 and the blood analyte sensor 22, including combinations of valves, lines, pipes, tubes, channels, and other direct or indirect connections.

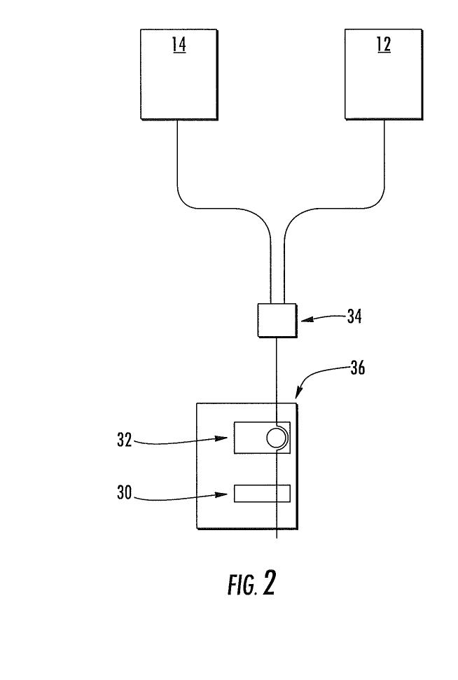

The calibrant sensor 20 in one embodiment, as shown in FIG. 2, may be contained within a measurement module 30 that is positioned downstream along the fluid column from a pump module 32 and a mixing module 34. In this embodiment, a consolidated flow control housing 36 includes both the measurement module 30 and the pump module 32 operated in a consolidated assembly allowing them to share flow control and computational resources. The upstream mixing module 34 is configured to ensure thorough mixing of the calibrant and diluent from the sources 12, 14.

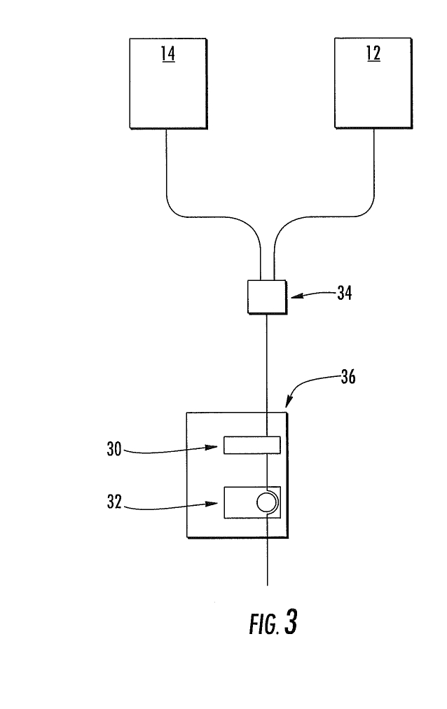

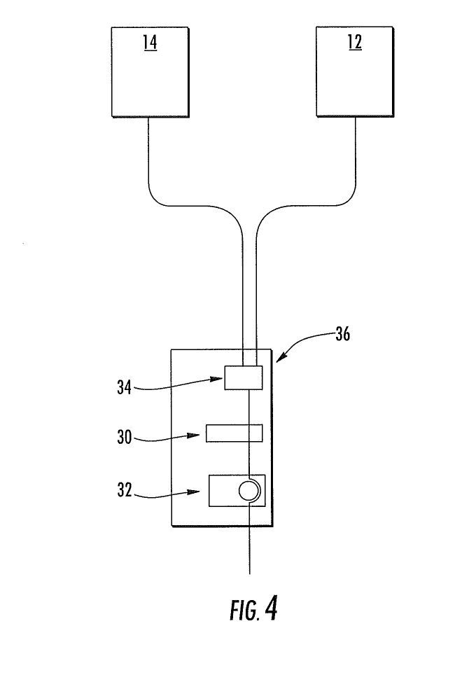

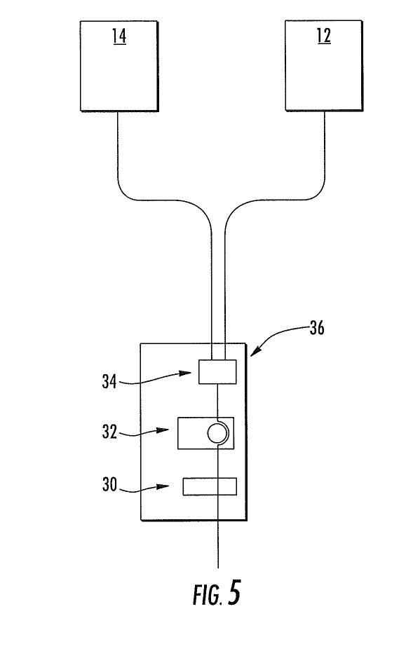

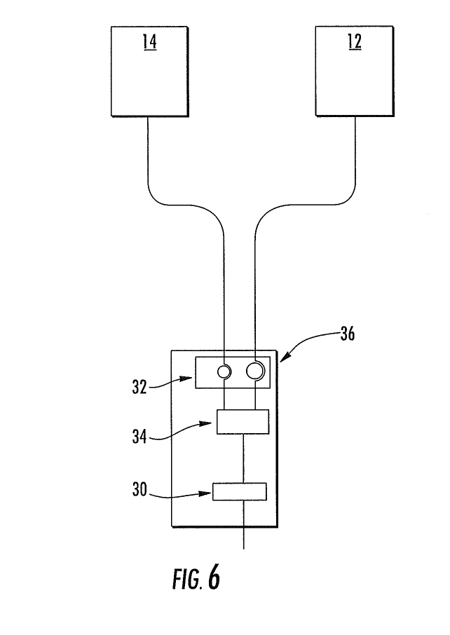

In another embodiment, as shown in FIG. 3, the measurement module 30 is upstream of the pump module 32, although both modules are still contained within the same flow control housing 36. In yet another embodiment, as shown in FIG. 4, the mixing module 34 has also been consolidated into the flow control housing 36. FIG. 5 shows another embodiment where the modules 30, 32, 34 are all consolidated within the flow control housing 36, but the measurement module 30 is the most downstream. In another embodiment, as shown in FIG. 6, the pump module 32 includes two rotary pinch valves that separately meter out calibrant and diluent from the sources 12, 14.

Preferably, the measurement module 30 and calibrant sensor 20 are configured to be low-cost and compact, but still to measure accurately the calibrant concentration of a relatively simple, clear (or less translucent than blood) solution in near real-time as it flows to the blood analyte sensor 22 during calibration cycles. As compared to the blood analyte sensor 22, therefore, the calibrant sensor 20 should generally be more accurate although it need not have as robust an ability to differentiate between the calibrant concentration and interferants that are in larger quantities in blood. Different types of calibrant sensor systems can be employed with a range of advantages and tradeoffs.

Examples of different embodiments for the calibrant sensor 20 (which can include a whole system of components to accomplish the “sensor” function) include polarization (polarimetry), refractometry, spectroscopy, optical and fluid density measurement (e.g., ultrasound techniques), viscosity, electrical impedance, or specific heat of the calibrant.

As shown in FIG. 7, a polarimetry system 38 of one embodiment includes a light source 40, a collimating lens 42, a polarizer 44, a liquid crystal polarization rotator (LCPR) 46, a sample chamber 48, an analyzer 50, a focusing lens 52 and a light detector 54. A polarimetry sensor operates under the principal that a chiral molecule (such as glucose in solution) refracts the two components of polarized light differently, thus altering the plane of polarization. Optical rotation at a single wavelength of polarized light can be correlated to known standards for optical rotation as a function of concentration for the chiral molecule of interest.

The light source 40 preferably emits single-wavelength light to simplify the correlation with concentration. For example, the light source 40 may be a laser light-emitting-diode (LED) that is within the blue (405 nm) through green (525 nm) spectrum. Other or multiple wavelengths can be employed for better differentiation of interferants and noise. For example, red at approximately 633 nm could be additionally employed for better results. This un-polarized monochromatic light is emitted to the collimating lens 42 which aligns the light into parallel waves for passage through the polarizer 44. The polarizer 44 is configured to orient all of the waves into a plane of polarized light.

The LCPR in one embodiment is a nematic liquid crystal retarder that has a substrate material including optical quality synthetic fused silica (Meadowlark Optics, Frederick, Colo.). Preferably, the resolution of the rotator is at least as fine as 1 m-degree due to the expected small rotation of polarity from the chiral optical characteristics of the dextrose calibrant in a saline solution.

Other options are available for pre-rotating the polarized laser light, such as a Faraday rotator (which uses doped crystals that respond to EM radiation), Pockels cells (which use electric fields) or manual or mechanical rotators (stepper motors) that change the rotation of the polarizer 44 to compensate for the rotation effect of the calibrant solution. An advantage of heterodyne systems over mechanical systems is the avoidance of moving parts and the finer achievable angular resolution, the latter of which is important to cutting down on the needed path length of the light through the sample. Some crystals that may be effective in the role of a retarder include lithium niobate, lithium tantalate, KDP and barium titanate.

As shown in FIG. 7, the sample chamber 48 includes an inlet 56, an outlet 58 and a central housing 60. The inlet 56 is an opening configured to receive attachment of one of the supply lines 16 which contains the calibrant in solution. The central housing 60 defines an opening or chamber that is configured to hold sufficient quantities of the calibrant solution (i.e., a sufficiently long path length such as 1 cm, 5 cm or 10 cm) to register a rotation of a magnitude within the resolution of the LCPR 46 and other components of the polarimeter calibrant sensor 20. At the same time, compact construction may be desired wherein the length of the sample chamber 48, or the entire sensor 20, or the total path length of the light through the sample, is 5 cm or less in length, or even 1 cm or less in length. Since the magnitude of the optical rotation due to a sample of glucose is directly proportional to the path length of the sample chamber, the main factor influencing the minimum path length of the sample chamber is the resolution of the measurement technique utilized in quantifying the optical rotation. Hence, if a given technique for measuring optical rotation allows a finer measurement resolution, the path length of the sample chamber can be made smaller.

The central housing 60 is constructed of a transparent plastic (e.g., polycarbonate or acrylic (PMMA)), crystal or other transparent or semi-transparent material that allows passage of the polarized, collimated laser light on its way from the light source 40 to the light detector 54. An area of concern for the use of these materials is that they may cause birefringence of the light passing through them and distort the results of the measurement. These birefringence characteristics may, for example, be due to the intrinsic nature of the material or stresses that they're under from being fashioned into the central housing 60. Birefringence may be controlled from sample chamber to sample chamber, making it a negligible issue. However, it may be a problem when it is stress-induced. In this situation, two sample chambers held by the same pump will give different optical rotations for a given glucose sample. Compensation for this error is possible if a “blank” measurement in air or saline can be made to determine and correct for disposable-to-disposable birefringence. From another perspective, disposable birefringence introduces an “offset” into the measurement. This offset needs to be tightly controlled through design and manufacturing, or corrected for by making a blank measurement.

The analyzer 50 is another (fixed) polarizer that is perpendicular to the light exiting the first polarizer 44 so as to block more of the exiting light when the LCPR 46 is at the proper, matching angle for the analyte concentration in the sample chamber 48. Light that is not blocked passes the analyzer 50 and shines through the focusing lens 52 on the light detector 54, such as a photodiode. This signal is used to adjust rotation of the LCPR 46 until the detected light is minimized, yielding the angle of rotation of the calibrant. Concentration measurements are thus conducted by applying voltages to the LCPR 46 and measuring the angle of polarization that maximizes the extinction of the polarized light passing through to the focusing lens 52 and the light detector 54. These voltages are correlated to the angle of polarization and that angle is correlated to established concentration standards for the analyte.

The inventors have observed that management of temperature-induced errors is beneficial to the polarimetry system 38. For example, variations of up to 140 m-degree of rotation per degree C. have been observed in experimental testing. Combinations of precision thermal control and recalibration processes can improve accuracy.

Precise thermal inputs with heating or cooling, such as through thermoelectric heating and cooling, may be employed. Precision thermal control can be facilitated by reducing the thermal mass of the LCPR 46. Temperature variations can also be dampened using a substantial thermal mass, such as steel, copper or aluminum, in contact with the LCPR.

Recalibration, such as through closed-loop feedback, wherein the LCPR could be re-zeroed with a non-polarization rotating input, such as air or lactated ringers solution in a sample chamber, could be employed. Also, drift in the LCPR rotation angle could be optically monitored by splitting the output of the LCPR and analyzing the output signal in a second reference channel, such as with a second analyzer and detector (and possibly employing the use of a second sample chamber filled with air or lactated ringer's solution.

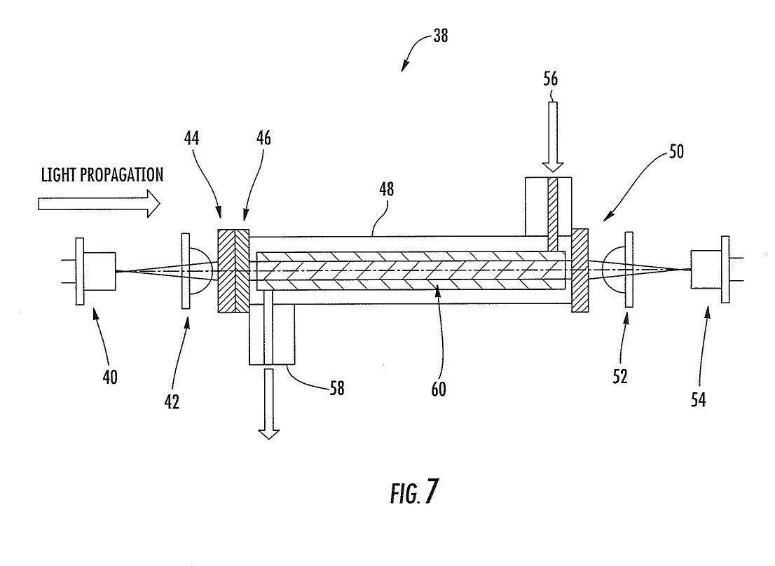

In another embodiment, as shown in FIG. 8, the polarimetry system 38 includes two Faraday based electro-optical rotators in a closed-loop digital control configuration. Closed-loop digital control configurations detect optical rotation directly and thus exhibit very stable long-term calibrations. They're also able to use low-cost laser sources, such as laser diodes, with similar sensitivity to other rotators. The polarimetry system 38 in FIG. 8 includes a laser 62, a polarizer 64, a Faraday modulator 66, a sample cell 68, a Faraday compensator 70, an analyzer 72, a photo detector 74, an amplifier 76, a line driver 78, a computer 80, and a digital lock-in amplifier 82.

The Faraday-based polarimetry disclosed herein is able to achieve sub-millidegree sensitivity. The laser 62 is a single laser and its light is passed through polarizer 64, which is a linear polarizer. The polarizer may include, for example, a film-type polarizer or a Glan-Thomspon or Glan-Taylor polarizer with about a 100,000:1 extinction ratio. Coupled to the polarizer 64 is the amplifier 76 which acts as the function generator, modulating the polarization vector by approximately +/−1 degree. The polarizer 64 and amplifier can be in turn coupled to the Faraday modulator 66 or they could be parts of the Faraday modulator itself. Further reduction in footprint also could result from using a single Faraday rotator as both the modulator and the compensator.

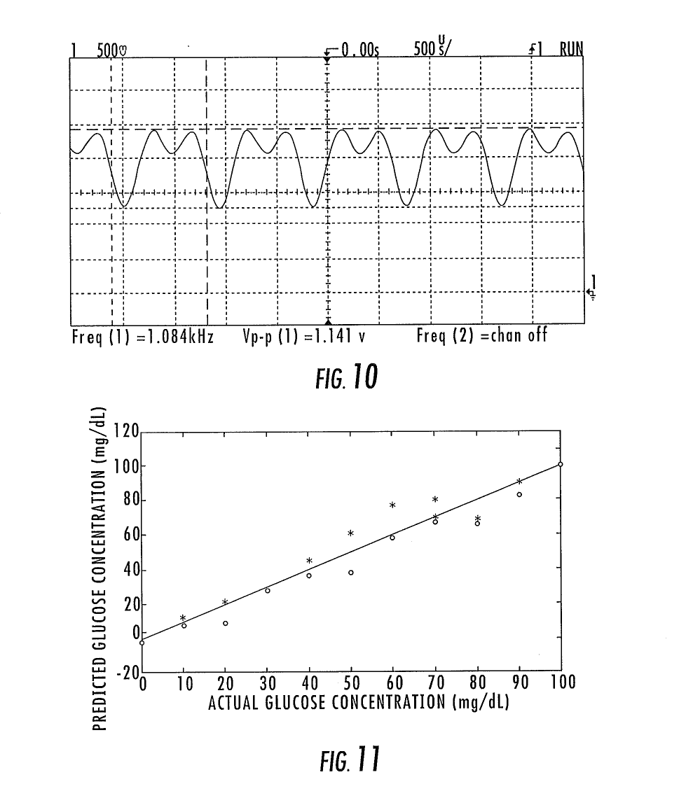

The lock-in amplifier 82 generates a sinusoidal reference signal, which is amplified by amplifier 76 and then passed to modulator 66. After optical modulation, the signal passes through the test or sample cell 68, which is filled with the calibrant solution and causes the signal to be modulated asymmetrically, as shown in FIG. 10. The illustrated signal contains both ωₘ and 2ωₘ components (where ωₘ is the Faraday rotator modulation frequency) due to the presence of glucose. The signal then passes through the analyzer 72, which is a polarizer initially oriented orthogonally to the input polarizer 64. What remains of the signal is then picked up by the photo detector 74, which in this embodiment is a simple Si-based detector.

The lock-in amplifier 82 is configured to receive the light signal from the photo detector 74 and measure the phase-sensitive amplitude of the single frequency component. This amplitude is proportional to the rotation of the light signal due to the glucose or analyte sample in the sample cell 68. The computer 80 and line driver 78 are configured to receive the amplitude information and act as a digital controller to direct the Faraday compensator 70 to rotate in response to the amplitude and “perfectly null” the system, i.e., negate the optical rotation of the sample (thus zeroing the ωₘ component of the detector signal and consequently the lock-in amplifier output).

FIG. 13 shows a more detailed schematic or variation of amplification and includes the photodetector 74's output sent to a photodetector amplifier, the lock-in amplifier 82, the power amplifier 76 and then the modulator 66. The lock-in amplifier 82 may include both a detector signal input and a reference signal output.

The digital controller, for example, may include a PID-type software-based controller. In discrete mode, the response of the polarimetry system 38 has a response time that is dependent on alignment of systems components and controller settings. If configured with proper PID settings, output stability for an accurate discrete measurement can usually be achieved in less than 2 seconds. More continuous mode operation is suited for flow-through sample cells 68 (likely the case for most embodiments of the present invention) because the lack of sample removal avoids perturbation.

In the embodiment of FIG. 8, the voltage required to null the system is the measured parameter of interest and is proportional to the glucose concentration. Mathematically, the operation of the system is described by an equation (1):

$$I ∝ {E}^{2} = ( {φ}^{2} + \frac{{θ}_{m}^{2}}{2} ) + 2 φ {θ}_{m} sin ( {ω}_{m} t ) - \frac{{θ}_{m}^{2}}{2} cos ( 2 {ω}_{m} t )$$

$$D C offset 1.09 kHz , {ω}_{m} 2.18 kHz , 2 {ω}_{m}$$

where θₘ is the depth of the Faraday modulation, ωₘ, is the modulation frequency, and φ represents the rotation due to the optically active sample subtracted by any feedback rotation due to the compensation Faraday rotator.

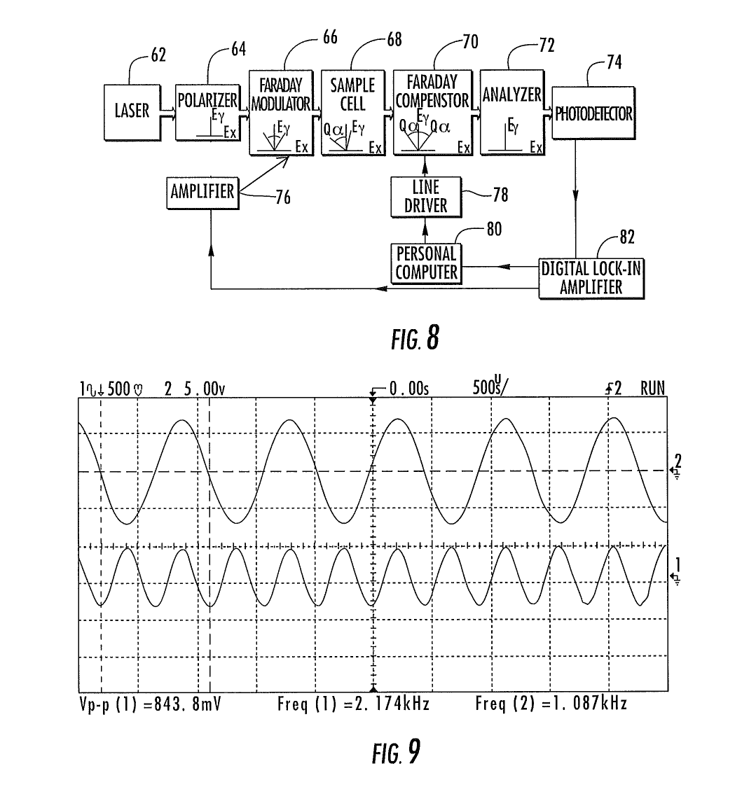

From equation (1) it is evident that without an optically active sample and with the DC term removed the detected signal only includes the double modulation frequency (2ωₘ) term, represented by the 2.18 kHz, 2ωₘ bottom signal in FIG. 9. However, when an optically active sample such as glucose is present, the detected signal then becomes an asymmetric sinusoid, for example as shown in FIG. 10, which contains both the fundamental (ωₘ) and the 2ωₘ modulation frequency terms.

As derived in equation (1), the signal from this single wavelength polarimeter is based on three terms; a DC term, a fundamental frequency term, and a double frequency term. It is the second amplitude parameter in the fundamental frequency term (ωₘ), that contains the glucose information in the angle φ. The feedback voltage applied to the Faraday compensator 70 from the line driver 78 is used to quantify the glucose.

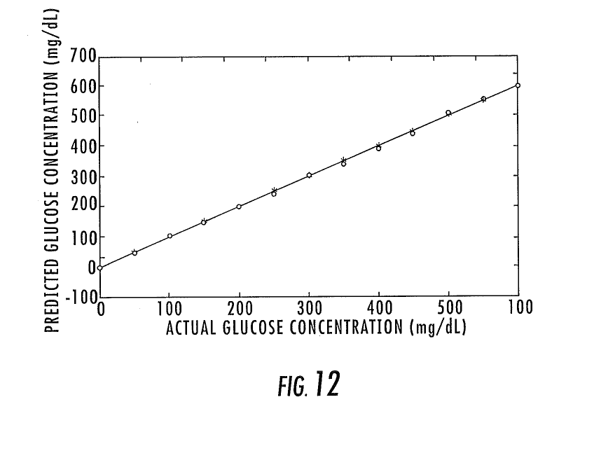

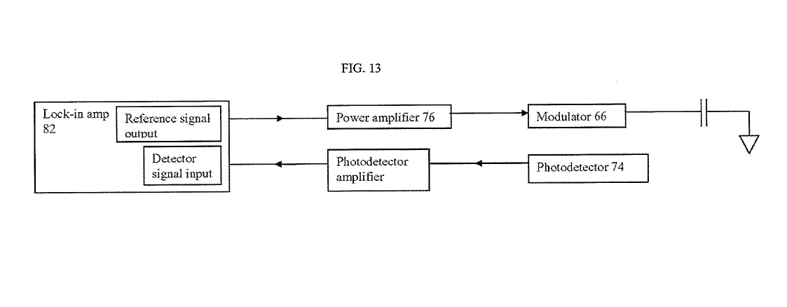

The embodiment of FIG. 8 was calibrated and validated. The system calibration in validation for a total of four individual runs—two using a 0-100 mg/dL range and two using a 0-600 mg/dL range—of glucose doped water are presented in FIGS. 11 and 12 with respective errors. In FIG. 11, for a 0-100 mg/dL range the ‘o’ has an SEP of 5.16 mg/dL and the ‘*’ has an SEP of 7.96 mg/dL. In FIG. 12, for a 0-600 mg/dL range the ‘o’ has an SEP of 5.78 mg/dL and the ‘*’ has an SEP of 4.34 mg/dL. All results show standard errors corresponding to better than 8% accuracy for a 670 nm laser diode and 1 cm path length.

This error could be reduced to within a 3-5% error for glucose concentrations in the 50 to 250 mg/dL range, such as by extending the path length to greater than 1 cm or using a lower wavelength laser. For instance, increasing the path length from 1 cm to 5 cm can result in a 5× increase in specific rotation of glucose. Moving from red to blue wavelength can result in a 2× increase in specific rotation of glucose, but may also cause increased sensitivity to stress-induced birefringence.

The method and apparatus for determining an analyte concentration in a patient's blood, or method and apparatus for determining calibrant concentration, such as set forth in the accompanying FIGS. 1 and 8, may be embodied by a computer program product. The computer program product includes a computer-readable storage medium, such as the non-volatile storage medium, and computer-readable program code portions, such as a series of computer instructions, embodied in the computer-readable storage medium. Usually, the computer program is stored by a memory device, such as RAM, and executed by an associated processing element, such as those contained in the monitor 24 shown in FIG. 1 or the computer 80 shown in FIG. 8.

In this regard, the figures of the present application include schematics of apparatus and program products according to exemplary embodiments of the invention. It will be understood that each element of the steps performed by such apparatus or programs can be implemented by computer program instructions. These computer program instructions may be loaded onto a computer or other programmable apparatus to produce a machine, such that the instructions which execute on the computer or other programmable apparatus provide means for implementing the functions described herein.

These computer program instructions may also be stored in a computer-readable memory that can direct a computer or other programmable apparatus to function in a particular manner, such that the instructions stored in the computer-readable memory produce an article of manufacture including instruction means which implement the functions performed by the apparatus. The computer program instructions may also be loaded onto a computer or other programmable apparatus to cause a series of operational steps to be performed on the computer or other programmable apparatus to produce a computer implemented process such that the instructions which execute on the computer or other programmable apparatus provide steps for implementing those functions.

Advantages of the analyte measurement system 10 of embodiments of the present invention include avoidance of the logistical issues associated with custom or pharmacy-created bags of calibrant solution. Instead, the end user has flexibility in creation of calibrant solutions with a range of technologies, none of them requiring controlled mixing with high accuracy. Also, the system could provide a safety enhancement through a confirmation of the concentration of a calibrant in real-time, avoiding any degradation of the calibrant solution concentration over time. Further, increased accuracy in the determination of the calibrant could improve the accuracy of calibration and analyte sensing in blood. The polarimetry system also has the advantages of being relatively compact and low-cost to create, while still affording robust operation and sufficient accuracy for the application.

Many modifications and other embodiments of the invention set forth herein will come to mind to one skilled in the art to which this invention pertains having the benefit of the teachings presented in the foregoing description. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

### REFERENCE NUMBERS

10 analyte measurement system
12 calibrant source
14 diluent source
16 fluid supply lines
18 pump
20 calibrant sensor
22 blood analyte sensor
24 monitor
26 pole
28 fluid control system
30 measurement module
32 pump module
34 mixing module
36 flow control housing
38 polarimetry system
40 light source
42 collimated lens
44 polarizer
46 LCPR
48 sample chamber
50 analyzer
52 focusing lens
54 light detector
56 inlet
58 outlet
60 central housing
62 laser
64 polarizer
66 Faraday modulator
68 sample cell
70 Faraday compensator
72 analyzer
74 photo detector
76 amplifier
78 line driver
80 computer
82 lock-in amplifier

That which is claimed:

1. A blood sensing system for sensing an analyte concentration of a patient's blood using a calibrant, the blood sensing comprising:
a blood sensor configured to generate a blood signal in response to exposure to the patient's blood;
a calibration system connected in communication with the blood sensor, and configured to record a calibration signal from the blood sensor in response to exposure to the calibrant; and
a calibrant concentration measurement system connected in communication with the calibration system and the blood sensor, the calibrant concentration measurement system including a calibrant sensor configured to measure a calibrant concentration in response to exposure to the calibrant,
wherein the calibrant concentration measurement system is further configured to use the calibrant concentration, calibration signal and the blood signal to determine the analyte concentration of the patient's blood.

2. A blood sensing system of claim 1, wherein the calibrant concentration measurement system is connected in fluid communication with the blood sensor and the calibrant passes through the calibrant concentration measurement system and to the blood sensor.

3. A blood sensing system of claim 2, wherein recording the calibration signal and measurement of the calibrant concentration occurs substantially contemporaneously.

4. A blood sensing system of claim 3, wherein measurement of the calibrant concentration occurs prior to recording of the calibration signal.

5. A blood sensing system of claim 4, wherein the calibrant concentration measurement system is connected in fluid communication with the blood sensor and is configured to receive and flow calibrant over the calibrant sensor and the blood sensor.

6. A blood sensing system of claim 5, further comprising a calibrant source.

7. A blood sensing system of claim 6, wherein the calibrant source is of a range of unknown concentrations.

8. A blood sensing system of claim 7, wherein the range of unknown concentrations is D5 to D50+/−5%.

9. A blood sensing system of claim 8, wherein the calibrant source includes at least two sources, wherein one of the sources is higher concentration and the other of relatively lower concentration.

10. A blood sensing system of claim 9, wherein the lower concentration is approximately zero.

11. A blood sensing system of claim 1, wherein the blood sensor is consumable and wherein the calibration system is configured to expose the blood sensor to the calibrant at regular intervals over a life of the blood sensor.

12. A blood sensing system of claim 11, wherein the blood sensor is consumable during less than a two-week period and the regular intervals are less than 10 minutes.

13. A blood sensing system of claim 1, further comprising a fluid control system connected in communication with both the calibration system and the calibrant concentration measurement system, the fluid control system configured to control exposure of the blood sensor and the calibrant sensor to the calibrant.

14. A blood sensing system of claim 13, wherein the fluid control system is further configured to control exposure of the blood sensor to the patient's blood.

15. A blood sensing system of claim 14, wherein the fluid control system includes a pump connected to a fluid column that includes an upper calibrant portion and a lower blood portion separated by a transition region.

16. A blood sensing system of claim 15, wherein the fluid column extends through the pump and the pump is configured to move the transition region above and below the blood sensor in cycles.

17. A blood sensing system of claim 16, wherein the calibrant sensor is positioned upstream of the blood sensor along the fluid column and the pump is further configured to stop movement of the transition region before it reaches the calibrant sensor.

18. A blood sensing system of claim 1, wherein the calibrant sensor uses one of polarization, refractometry, spectroscopy, density, viscosity, electrical impedance or specific heat of the calibrant to determine the calibrant concentration.

19. A blood sensing system of claim 1, wherein the calibrant sensor is a polarimeter including a light source, a first polarizer and a light detector.

20. A blood sensing system of claim 19, wherein the light source is a light-emitting-diode.

21. A blood sensing system of claim 20, wherein the light-emitting-diode is one of a green or a blue light-emitting diode.

22. A blood sensing system of claim 20, wherein light from the light-emitting diode is in a range of 405 nm to 525 nm wavelengths.

23. A blood sensing system of claim 19, wherein the first polarizer includes a rotator.

24. A blood sensing system of claim 23, wherein the rotator is configured to rotate polarized light to compensate for polarized light rotation by the calibrant.

25. A blood sensing system of claim 24, wherein the first polarizer and light detector are positioned on opposite sides of the calibrant.

26. A blood sensing system of claim 25, further comprising a second polarizer, wherein the first and second polarizers are positioned on opposite sides of the calibrant.

27. A blood sensing system of claim 26, wherein the second polarizer includes a rotator configured to rotate the polarized light to compensate for rotation of the polarized light by the calibrant.

28. A blood sensing system of claim 27, wherein the polarimeter includes a controller configured to communicate with the light detector and adjust rotation of the second polarizer to minimize detected light exiting the second polarizer.

29. A blood sensing system of claim 28, wherein the first polarizer is a Faraday modulator and the second polarizer is a Faraday compensator.

30. A blood sensing system of claim 29, wherein the calibrant sensor has an error of 5% or less for an 80-120 mg/dL range of calibrant concentration and a path length of less than 5 cm.

31. A blood sensing system of claim 29, further comprising a lock-in amplifier configured to provide a sinusoidal reference signal to the Faraday modulator.

32. A blood sensing system of claim 31, further comprising an amplifier configured to amplify the sinusoidal reference signal.

33. A blood sensing system of claim 32, wherein the amplifier is configured to lock-in to a fundamental frequency representing the Faraday modulator drive signal.

34. A blood sensing system of claim 29, wherein the first and second polarizers are liquid crystal polarizers.

35. A blood sensing system of claim 19, wherein the polarimeter includes a sample chamber through which the calibrant flows continuously.

36. A blood sensing system of claim 19, wherein the polarimeter includes a recalibration system configured to recalibrate the polarimeter based on measurements of a sample of known concentration.

37. A method of sensing an analyte concentration of a patient's blood using a calibrant, the method comprising:
generating a blood signal using a blood sensor in response to exposure to blood;
recording a calibration signal using the blood sensor in response to exposure to the calibrant;
measuring a calibrant concentration of the calibrant using a calibrant sensor in response to exposure to the calibrant; and
calculating the analyte concentration of the patient's blood using the calibrant concentration, calibration signal and the blood signal.

38. A method of claim 37, wherein recording the calibration signal and measuring calibrant concentration occur substantially simultaneously.

39. A method of claim 38, wherein measuring the calibrant concentration occurs prior to recording the calibration signal.

40. A method of claim 39, further comprising flowing calibrant over the calibrant sensor and then the blood sensor.

41. A method of claim 40, further comprising mixing a calibrant source and a diluent source to create calibrant.

42. A method of claim 41, wherein the method is cycled continuously for at least 72 hours.

43. A method of claim 41, further comprising exposing the blood sensor by drawing blood over the blood sensor.

44. A method of claim 43, further comprising moving a transition region between a calibrant portion of a fluid column and a blood portion of the fluid column above and below the blood sensor in cycles.

45. A method of claim 44, further comprising stopping movement of the transition region before it reaches the calibrant sensor.

46. A method of claim 37, wherein measuring the calibrant concentration includes transmitting polarized light through the calibrant and determining a rotation of the polarized light by the calibrant and correlating the rotation to a concentration.

47. A method of claim 46, wherein determining the rotation includes rotating the polarized light to offset rotation by the calibrant until a null signal is achieved.

48. A method of claim 47, wherein transmitting polarized light includes driving the polarization rotation with a sinusoidal signal.

49. A method of claim 48, wherein calculating the analyte concentration includes locking in to the frequency of modulation of the polarization rotator representing calibrant concentration.
